(19) United States
(12) Patent Application Publication
(10) Pub. No.: US 2025/0072969 A1
(43) Pub. Date: Mar. 6, 2025

(54) SYSTEMS AND METHODS FOR INTEGRATING INTRA-OPERATIVE IMAGE DATA WITH MINIMALLY INVASIVE MEDICAL TECHNIQUES

(71) Applicant: INTUITIVE SURGICAL OPERATIONS, INC., SUNNYVALE, CA (US)

(72) Inventors: Julie Walker, San Francisco, CA (US); Troy K. Adebar, San Jose, CA (US); Cristian Bianchi, Capannori (IT); Zachary Moller, San Francisco, CA (US); Leah Muller, San Francisco, CA (US); Cynthia Warman, San Francisco, CA (US); Sungwon Yoon, Palo Alto, CA (US)

(21) Appl. No.: 18/725,235

(22) PCT Filed: Dec. 27, 2022
(86) PCT No.: PCT/US2022/082437

§ 371 Date: Jun. 28, 2024

Related U.S. Application Data
(60) Provisional application No. 63/295,701, filed on Dec. 31, 2021.

(51) Int. Cl. A61B 34/10; A61B 34/00; A61B 90/00

(52) U.S. Cl. CPC A61B 34/10; A61B 34/25; A61B 90/37; A61B 2034/105; A61B 2034/107; A61B 2090/3764

(57) ABSTRACT

A system comprises a processor, a display, and a memory having computer readable instructions stored thereon that, when executed by the processor, cause the system to receive intra-operative three-dimensional image data from an imaging system. A portion of the intra-operative three-dimensional image data corresponds to an instrument disposed in a patient anatomy. The computer readable instructions, when executed by the processor, further cause the system to generate two-dimensional projection image data from the intra-operative three-dimensional image data, display the two-dimensional projection image data on the display, and identify, within the two-dimensional projection image data, a three-dimensional location of a portion of the instrument.

### CROSSED-REFERENCED APPLICATIONS

This application claims priority to and benefit of U.S. Provisional Application No. 63/295,701, filed Dec. 31, 2021 and entitled “Systems and Methods for Integrating Intra-Operative Image Data With Minimally Invasive Medical Techniques,” which is incorporated by reference herein in its entirety.

### FIELD

The present disclosure is directed to systems and methods for planning and performing an image-guided procedure.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Medical tools may be inserted into anatomic passageways and navigated toward a region of interest within a patient anatomy. Navigation may be assisted using images of the anatomic passageways, obtained pre-operatively and/or intra-operatively. Improved systems and methods are needed to enhance procedure workflow by coordinating medical tools and images of the anatomic passageways.

### SUMMARY

Consistent with some examples, a system may comprise a processor, a display, and a memory having computer readable instructions stored thereon that, when executed by the processor, cause the system to receive intra-operative three-dimensional image data from an imaging system. A portion of the intra-operative three-dimensional image data corresponds to an instrument disposed in a patient anatomy. The computer readable instructions further cause the processor to generate two-dimensional projection image data from the intra-operative three-dimensional image data, display the two-dimensional projection image data on the display, and identify, within the two-dimensional projection image data, a three-dimensional location of a portion of the instrument.

In some examples, the computer readable instructions, when executed by the processor, may cause the system to segment, based on the identified three-dimensional location of the portion of the instrument, the portion of the intra-operative three-dimensional image data corresponding to the instrument. The computer readable instructions, when executed by the processor, may cause the system to register the intra-operative three-dimensional image data to shape data from the instrument by comparing the shape data to the portion of the intra-operative three-dimensional image data corresponding to the instrument and display a two-dimensional projection of the shape data on the two-dimensional projection image data. The computer readable instructions, when executed by the processor, may cause the system to identify one or more regions of the shape data that is misaligned with the portion of the intra-operative three-dimensional image data corresponding to the instrument and display the one or more regions with at least one visual property different than one or more regions of the shape data that is aligned with the portion of the intra-operative three-dimensional image data corresponding to the instrument. The at least one visual property may comprise at least one of a color, a brightness, a linetype, a pattern, or an opacity.

In some examples, the computer readable instructions, when executed by the processor, may cause the system to receive a user input and, based on the user input, adjust at least one of a position or a rotation of the shape data with respect to the intra-operative three-dimensional image data.

In some examples, the computer readable instructions, when executed by the processor, may cause the system to generate a model of the patient anatomy based on pre-operative image data and update the model based on the intra-operative three-dimensional image data. Updating the model may include revising a location of an anatomical target. The computer readable instructions, when executed by the processor, may cause the system to generate a navigation path through the patient anatomy based on the pre-operative image data. Updating the model may include revising the navigation path to correspond to the revised location of the anatomical target. The computer readable instructions, when executed by the processor, may cause the system to generate a model of the patient anatomy based on pre-operative image data and register the model to the intra-operative three-dimensional image data based at least in part on a location of an anatomical target in each of the model and the intra-operative three-dimensional image data. The computer readable instructions, when executed by the processor, may cause the system to extract a three-dimensional boundary of an anatomical target from a model of the patient anatomy generated based on pre-operative image data and display a projection of the three-dimensional boundary of the anatomical target on the two-dimensional projection image data. The computer readable instructions, when executed by the processor, may cause the system to receive an input from a user to manipulate at least one of a location or a dimension of the projection of the three-dimensional boundary.

In some examples, the imaging system may comprise a cone-beam computed tomography system. The two-dimensional projection image data may comprise at least one maximum intensity projection of the intra-operative three-dimensional image data based on voxel intensity values. Displaying the two-dimensional projection image data on the display may include displaying a plurality of views with different view orientations. The plurality of views may include at least a first view and a second view. An orientation of the first view may be orthogonal to an orientation of the second view. Each of the first view and the second view may include one of an axial view, a coronal view, or a sagittal view. Identifying the three-dimensional location of the portion of the instrument may include receiving a first user input indicating a first two-dimensional location of the portion of the instrument in the first view and receiving a second user input indicating a second two-dimensional location of the portion of the instrument in the second view.

In some examples, the computer readable instructions may, when executed by the processor, cause the system to select a region of interest within the intra-operative three-dimensional image data based on the identified three-dimensional location of the portion of the instrument, generate two-dimensional projection image data from the selected region of interest within the intra-operative three-dimensional image data, and display the two-dimensional projection image data from the selected region of interest on the display.

Displaying the two-dimensional projection image data on the display may include displaying a first view with a view plane having a view plane orientation. Identifying the three-dimensional location of the portion of the instrument may include receiving a user input indicating a location of the portion of the instrument in the first view, wherein the indicated location is identifiable by a first coordinate value and a second coordinate value of respective orthogonal first and second axes within the view plane, and identifying a third coordinate value associated with the indicated location by retrieving a stored coordinate value of a voxel producing a maximum intensity at the indicated location of the portion of the instrument in the first view. The third coordinate value may represent an axis orthogonal to the view plane orientation. The portion of the instrument may be a distal tip of the instrument. The distal tip may be constructed from a material associated with a high intensity value relative to anatomical tissue, such as a material associated with high Hounsfield unit values relative to anatomical tissue.

In some examples, the system may further include the imaging system and/or the instrument.

Consistent with some examples a method may comprise registering shape data from an instrument disposed in a patient anatomy to a model of the patient anatomy, wherein the model of the patient anatomy includes an anatomical target, displaying the shape data in relation to the model of the patient anatomy on a display, obtaining intra-operative three-dimensional image data with an imaging system, wherein the intra-operative three-dimensional image data includes at least a portion of the instrument and the anatomical target, measuring a relationship between the portion of the instrument and the anatomical target in the intra-operative three-dimensional image data, and revising a location of the anatomical target in the model of the patient anatomy so that a relationship between a portion of the shape data corresponding to the portion of the instrument and the location of the anatomical target in the model of the patient anatomy corresponds to the measured relationship between the portion of the instrument and the anatomical target in the intra-operative three-dimensional image data.

In some examples, measuring the relationship between the portion of the instrument and the anatomical target in the intra-operative three-dimensional image data may include measuring at least one of a distance or an orientation between the portion of the instrument and the anatomical target in the intra-operative three-dimensional image data.

In some examples, measuring the relationship between the portion of the instrument and the anatomical target in the intra-operative three-dimensional image data may include determining an offset between the portion of the instrument and the anatomical target in the intra-operative three-dimensional image data. The offset may include an x-distance, a y-distance, and a z-distance corresponding to respective axes.

In some examples, revising the location of the anatomical target in the model of the patient anatomy may include receiving, via user input, the x-distance, the y-distance, and the z-distance and moving the location of the anatomical target in the model of the patient anatomy to a location offset from the portion of the shape data by the x-distance, the y-distance, and the z-distance.

Other examples include corresponding computer systems, apparatus, and computer programs recorded on one or more computer storage devices, each configured to perform the actions of the methods.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 illustrates a display system displaying an image of an instrument registered to a model.

FIG. 2 illustrates an example of a method or workflow for performing a minimally invasive procedure using an integrated imaging system in accordance with some aspects of the present disclosure.

FIGS. 3A-3C illustrate a graphical user interface incorporating features of the present disclosure.

FIG. 4 illustrates a simplified diagram of registering pre-operative image data and intra-operative image data to shape data from an instrument.

FIG. 5 illustrates an example of a method or workflow for performing a minimally invasive procedure using a non-integrated imaging system in accordance with some aspects of the present disclosure.

FIGS. 6A-6C illustrate a graphical user interface incorporating features of the present disclosure.

FIG. 7A illustrates a simplified diagram of a robot-assisted medical system according to some examples.

FIG. 7B illustrates a simplified diagram of communication between a control system and an intra-operative imaging system.

FIG. 8 illustrates a simplified diagram of an instrument system and an intra-operative imaging system according to some examples.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

The techniques disclosed in this document may be used to enhance the workflow processes of procedures discussed herein, including minimally invasive procedures, using intra-operative imaging, such as cone beam computerized tomography (CT) imaging. Although described primarily in the context of medical procedures, it should be appreciated that this disclosure has non-medical applications as discussed throughout.

In some examples, including an “integrated” system in which an instrument control system is in operative communication with an intra-operative imaging system for transfer of intra-operative image data to the instrument control system, instrument shape data may be used to register a pre-operative three-dimensional (“3D”) model of patient anatomy to intra-operative images received at the instrument control system from the intra-operative imaging system. In some examples, including a “non-integrated” system in which an instrument control system is not in operative communication with an intra-operative imaging system, a target location with respect to an instrument as determined in intra-operative images on the intra-operative imaging system, may be used to update a target location in a pre-operative three-dimensional model of patient anatomy using the instrument control system. In both integrated and non-integrated imaging systems, the image data produced by intra-operative imaging may be utilized to refine locations of targets in a model constructed from pre-operative imaging.

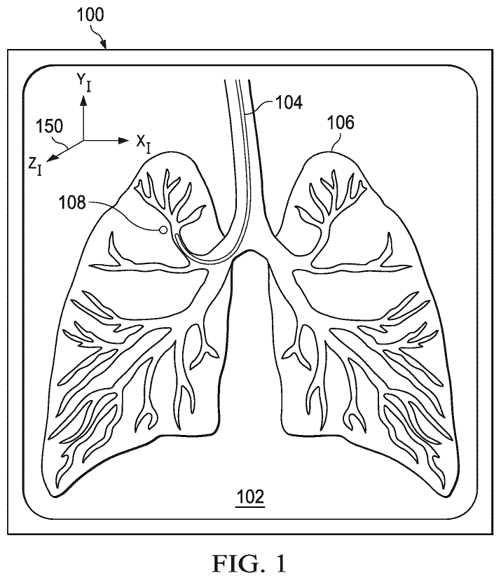

With reference to FIG. 1, an image-guided procedure, which may be robot-assisted or otherwise teleoperated, may be conducted in which a display system 100 may display a virtual navigational image 102, having an image reference frame (X_{I}, Y_{I}, Z_{I}) 150 in which an instrument 104 is registered (e.g., dynamically referenced) with a model 106, which may be a model of a patient derived from pre-operative image data obtained, for example, from a CT scan. As non-limiting examples, an instrument as that term is used herein may include a catheter, an endoscope, graspers, scissors, a cautery device, an ablation tool, a diagnostic or therapeutic needle, a stapler, an ultrasound probe, or any other suitable imaging probe or medical or non-medical instrument.

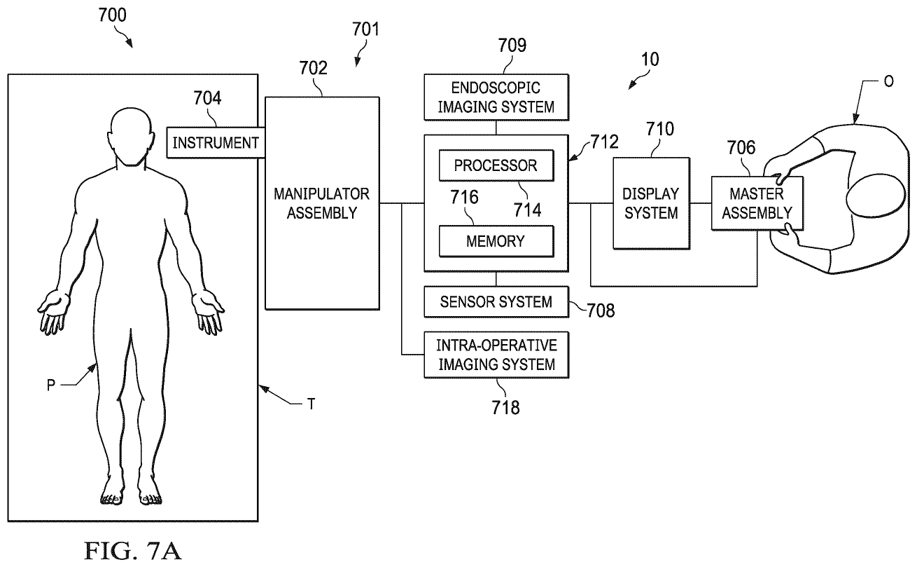

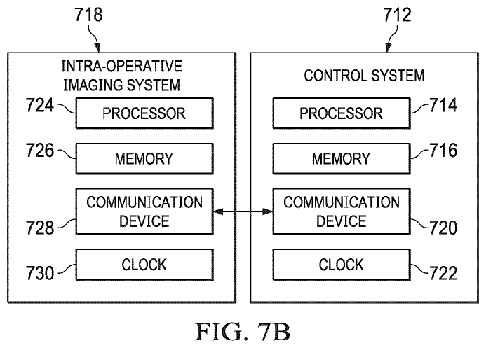

The model 106 may include a target 108, such as a lesion, nodule, or other structure of interest in a patient anatomy or other environment, which the procedure is intended to address (e.g., biopsy, treat, explore, view, etc.). In some examples, the virtual navigational image 102 may present a user with a virtual image of the internal environment site from a viewpoint of the instrument 104. In some examples, the display system 100 may present a real-time view from the distal tip of instrument 104, for example, when the instrument 104 comprises an endoscope. In some examples, the instrument 104 may be manipulated by a robot-assisted manipulator controlled by an instrument control system, or processing system, which includes one or more processors. An example of a robot-assisted system will be described further at FIG. 7A.

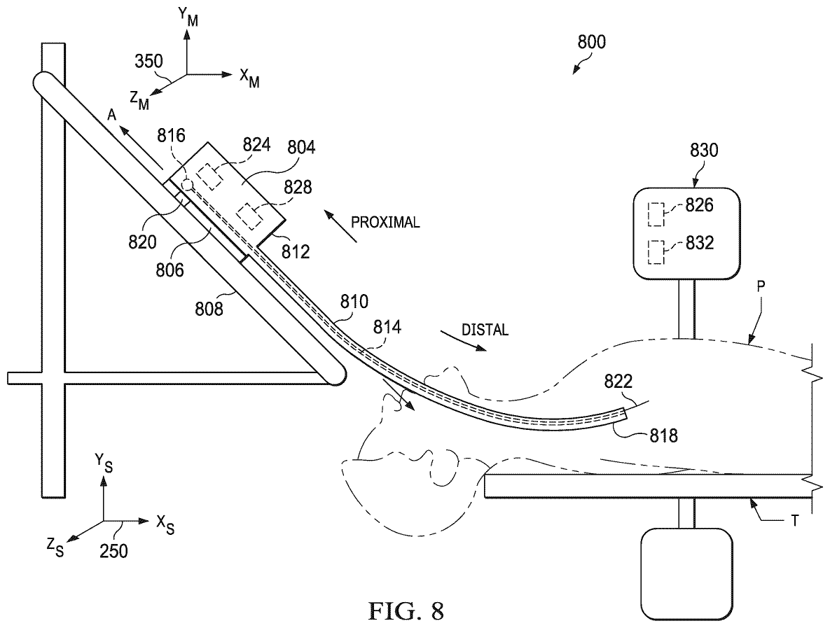

Generating the virtual navigational image 102 involves the registration of the image reference frame (X_{I}, Y_{I}, Z_{I}) 150 to a surgical reference frame (X_{S}, Y_{S}, Z_{S}) of the anatomy and/or a medical instrument reference frame (X_{M}, Y_{M}, Z_{M}) of the instrument 104, in medical examples. Examples of the surgical reference frame and medical instrument reference frame are shown in FIG. 8. This registration may rotate, translate, or otherwise manipulate by rigid or non-rigid transforms points associated with the segmented instrument shape from the image data and/or points associated with the shape data from a shape sensor disposed along a length of the instrument 104. This registration between the image and instrument reference frames may be achieved, for example, by using a point-based iterative closest point (ICP) technique as described in U.S. Pat. App. Pub. Nos. 2018/0240237 and 2018/0235709, incorporated herein by reference in their entireties, or another point cloud registration technique.

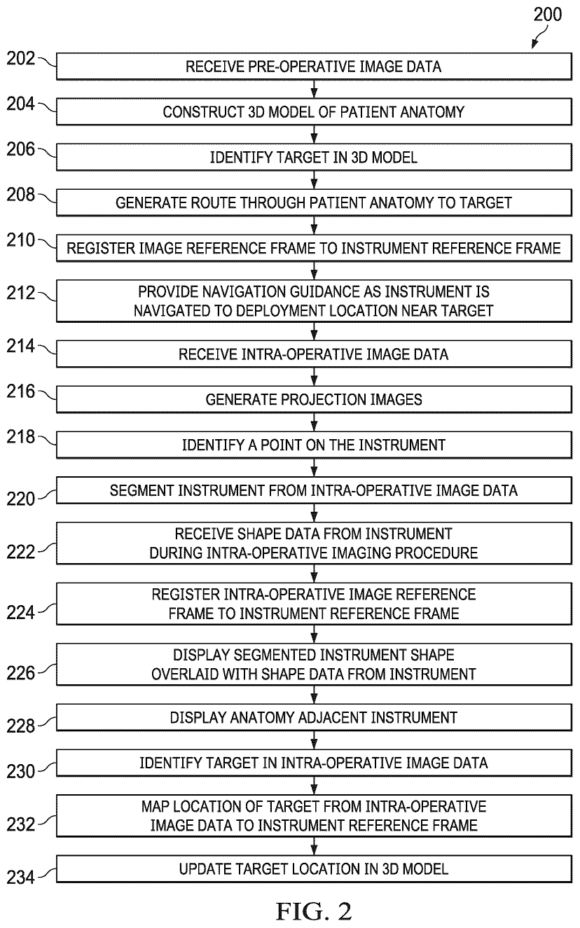

FIG. 2 illustrates an example of a method or workflow 200 for performing a minimally invasive procedure using an integrated imaging system in accordance with some aspects of the present disclosure. At a process 202, pre-operative image data is received at a control system. For example, a CT scan of the patient's anatomy may be performed with a conventional fan beam CT scanner, and the CT image data may be received by a control system. Alternatively, pre-operative image data may be received from other types of imaging systems including magnetic resonance imaging systems, fluoroscopy systems, or any other suitable method for obtaining dimensions of anatomic structures. At process 204, a three-dimensional (3D) model of the anatomic structures (e.g., model 106 of FIG. 1) may be constructed from the pre-operative image data by a control system. At process 206, a target may be identified in the 3D model or the pre-operative image data from which it was constructed. For example, the target 108 of FIG. 1 may be identified in the model 106 as a region of interest for investigation or treatment. The target may be automatically identified by a control system and confirmed by a user or may be visually identified by the user and manually selected or indicated in the 3D model, for example, through the display system 100. At process 208, a route through anatomic passageways formed in the anatomic structures is generated. The route may be generated automatically by the control system, and/or the control system may generate the route based on user inputs. The route may indicate a path along which an instrument (e.g., instrument 104 of FIG. 1) may be navigated to a deployment location in close proximity with the target. In some examples, the route may be stored in the control system and incorporated into the images displayed on display system 100.

To provide accurate navigation through the anatomic passageways, an image reference frame 150 of the pre-operative image data (and subsequently constructed 3D model) may be registered to an instrument reference frame of the instrument at process 210. For example, a shape sensor (e.g., a fiber optic shape sensor or one or more position sensors) disposed along a length of the instrument may be used to provide real-time shape data (e.g., information regarding a shape of the instrument and/or a position or orientation of one or more points along the length of the instrument). This shape data may be utilized to register the instrument to the 3D model constructed from the pre-operative image data and to track a location of the instrument with respect to the patient anatomy displayed in the 3D model during use. Upon successful registration, a process 212 may include providing navigation guidance as the instrument is navigated through the anatomic passageways to a deployment location in proximity to the target. In some examples, the deployment location may be the location of the target itself while in other examples the deployment location may be location near the target that is suitable for deployment or use of a tool from the instrument. Navigation may be performed manually by a user with navigation guidance provided by the control system, automatically by the control system, or via a combination of both.

With the instrument positioned at or near a deployment location, an intra-operative imaging scan may be performed. At a process 214, intra-operative image data may be received at an instrument control system from the intra-operative imaging system. In some examples, the intra-operative imaging system may be a cone beam CT (“CBCT”) scanner than generates intra-operative CT scan image data, although any suitable imaging technique may be used without departing from the examples of the present disclosure. As compared to other imaging techniques such as MRI, conventional CT, or fluoroscopy, CBCT imaging may provide a more rapid scan of a region of the patient's anatomy to minimize delay of the procedure and may also be available with hardware that is more portable and compact than other imaging modalities.

As mentioned above, in an integrated imaging system, the intra-operative image data may be received at a control system or other processing platform associated with the instrument. In some examples the data associated with the instrument, such as shape data, may be transferred from the instrument control system to the intra-operative imaging system, or both the shape data and the image data may be transferred to a common processing platform. In this regard, registration of the shape data of the instrument to the intra-operative image data may be performed by the instrument control system, by the imaging system, or by another platform in operable communication with the intra-operative imaging system and the instrument control system. As an example, the communication of the image data to or from a control system may use a Digital Imaging and Communications in Medicine (“DICOM”) standard. In some examples, the image data may include one or more timestamps associated with the image data for synchronizing instances of image data and shape data. A first timestamp may indicate the start time of the scan and a second timestamp may indicate a stop time of the scan. Alternatively, a separate timestamp may be associated with each image of the image data.

At process 216, the control system may generate one or more projection images from the intra-operative image data. A projection image may be a two-dimensional (“2D”) image created from and configured to represent 3D intra-operative image data. A projection image may be generated by selecting or averaging intensity values of voxels, which may represent an image brightness or other characteristic of a voxel, extending along a plurality of projection lines orthogonal to a viewing plane of the projection image, resulting in pixels across the viewing plane which each represent one or more voxels along a respective projection line. As an example, a maximum intensity projection image may be created by establishing a viewing plane and selecting a highest-value voxel along each projection line through the 3D intra-operative image data that is orthogonal to the viewing plane. Although any arbitrary viewing plane may be established, in some examples, a projection image is generated for each cardinal plane (e.g., sagittal, coronal, and axial) of the intra-operative image data. As an example, a projection image may be generated for an x-y view in which the highest-value voxel along the z-dimension through the 3D image data is selected and projected into a pixel in the viewing plane. A similar approach may also be used for each of the x-z and y-z views. In an alternative example, a viewing plane may be selected that provides an optimal viewing angle for a particular target. It should be appreciated that a “slice” or “slice image” as that term is used herein refers to a 2D image taken along an image plane extending through 3D volumetric image data such that a slice displays only features intersected by the image plane. In contrast, a “projection image” may include features from any number of different parallel image planes through the 3D volumetric image data.

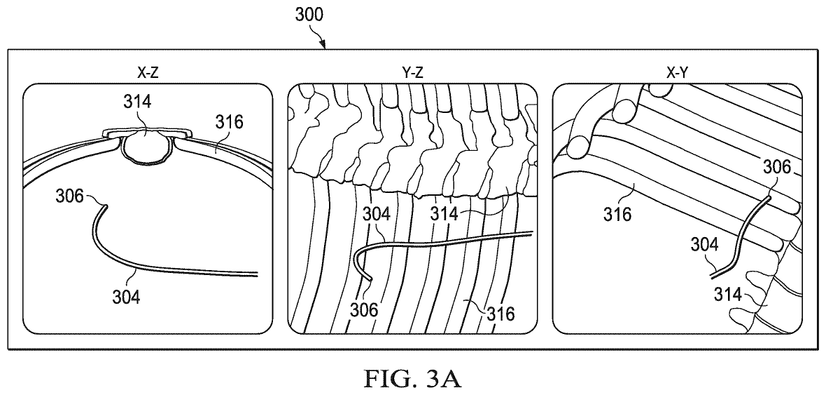

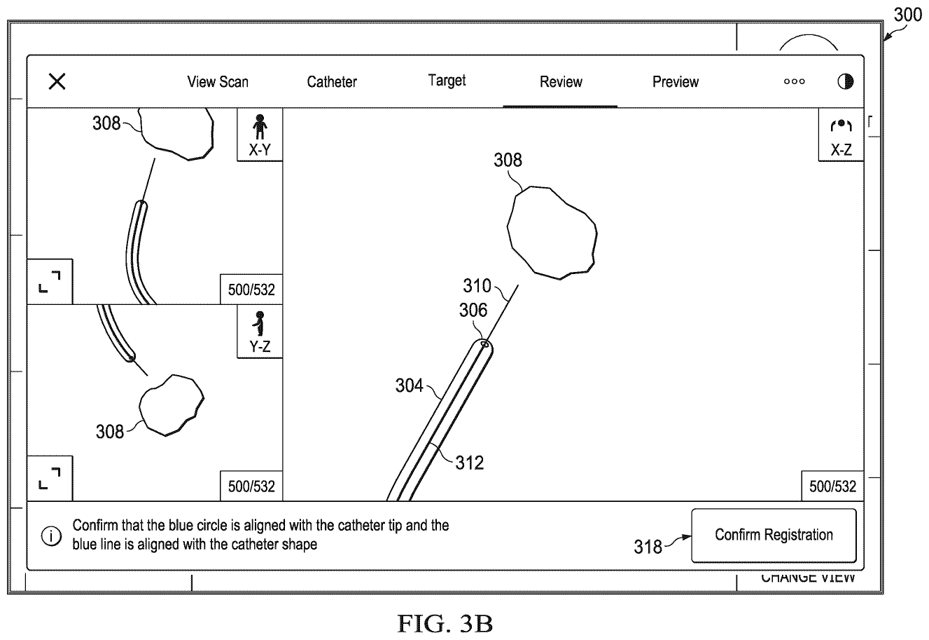

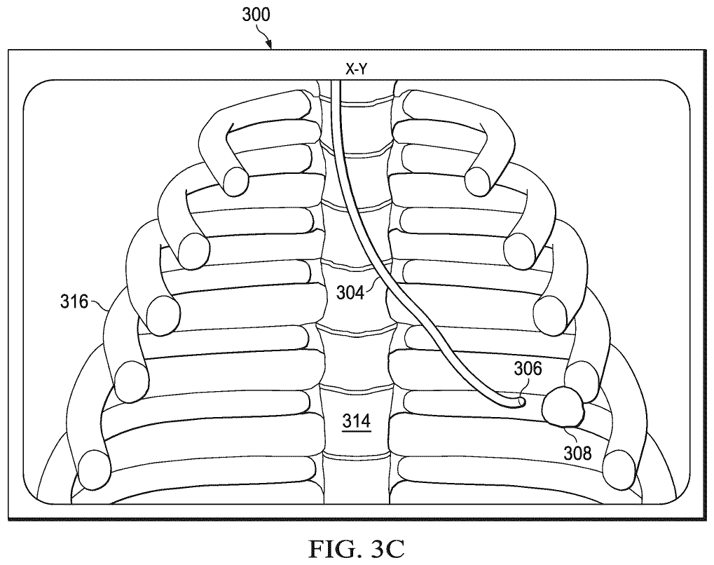

The highest-value voxel along a projection line may be the voxel associated with the highest intensity value (e.g., CT number or Hounsfield units). Because an instrument is typically constructed, at least in part, from metals and other materials that are denser than the surrounding anatomy, the instrument may appear to have a high intensity (e.g., brightness) in the intra-operative image data with a distinct contrast to its darker anatomical surroundings. Consequently, voxels representing the instrument are likely to be selected as the highest-value voxel along a projection line when generating a maximum intensity projection image. Accordingly, the resultant projection image is likely to include all or a substantial portion of the instrument present in the intra-operative image data displayed with a significant contrast to the surrounding anatomy. Thus, a projection image may provide for simplified visual identification of the instrument as compared to traditional volumetric image display techniques in which a user must scroll through image slices and identify only a small cross-sectional area of an instrument. In some examples, a distal tip of the instrument may be constructed from a material having a density greater than other portions of the instrument, causing the distal tip to appear as the brightest feature in the images for simplified visual identification of the distal tip. Examples of projection images are provided in FIG. 3A which is discussed further below.

Intra-operative image data may encompass a large volume of the patient anatomy, other tools positioned within the patient, and even structures external to the patient such as an operating table or external instrumentation. In order to avoid obscuring the instrument or tissue of interest in a projection image, one or more constraints, such as spatial limits or intensity thresholds, may be utilized when generating a projection image. For example, only a subset volume of the intra-operative image data in a region of interest around the instrument or target may be considered when generating a projection image. The size and location of the subset volume may be determined by a variety of factors including, but not limited to, a position of the instrument based on shape data from a shape sensor or a location of the instrument as determined in a prior projection image (e.g., identify tip in large projection image and then generate a smaller projection image around the tip to enhance visibility of tissues near the tip). Voxels of structures outside the region of interest may be disregarded when selecting a highest-value voxel along a projection line to generate a projection image. As another example, when an extraneous structure such as a metallic operating table or fiducial marker is known to result in voxel intensity values exceeding those of the instrument, a threshold value between that of the instrument and the operating table may be implemented to establish a maximum acceptable value when selecting a highest-value voxel. As a result, a voxel corresponding to the extraneous structure may be disregarded when selecting a highest-value voxel along a projection line passing through the instrument and the extraneous structure. Similar spatial limit and threshold techniques may be used to omit other high intensity tools, structures, and dense anatomical structures from a projection image. These techniques may allow the control system to generate projection images that include and more clearly visualize a substantial portion of the instrument for simplified identification of the instrument in the intra-operative image data or more clearly visualize anatomical structures near the instrument.

At process 218, a point on the instrument may be identified in the one or more projection images by a user via a user input device. Although any point along a length of the instrument may be identified and selected, a distal tip of the instrument may be constructed from a material, as discussed above, that appears with a high intensity in the intra-operative image data such that the distal tip may be easiest to identify.

A number of techniques for selecting a point on the instrument are contemplated. In some examples, a user can select an instrument point in one or more slice images of the intra-operative image data and the corresponding location of the selected point can be indicated by the control system with a marker on the one or more projection images for visual confirmation that the selected point is, in fact, on the instrument. Similarly, a user can select an instrument point on the one or more projection images and the corresponding location of the selected point can be indicated by the control system with a marker on the slices of the intra-operative image data for visual confirmation by the user that the selected point is on the instrument. A control system may allow a user to toggle between a slice image and a projection image having the same viewing orientation.

Because a projection image is a 2D representation of 3D image data, identifying a point in a single projection image will typically provide a location of the point along the two dimensions (e.g., vertical and horizontal) of the projection image, but not along the third dimension (e.g., depth) orthogonal to the projection image. A number of techniques are contemplated for obtaining a location of the point in three dimensions. For example, a user can select the same point in at least two different 2D projection views obtained at different viewing angles, preferably orthogonal to one another. For example, if a user selects the distal tip of the instrument in a first projection image having an X-Y viewing plane and in a second projection image having a Y-Z viewing plane, each of the X-, Y-, and Z-coordinates of the distal tip may be obtained from the two selections of the distal tip. However, it should be appreciated that any two non-parallel viewing planes could be used to determine a 3D location of a point. In some examples, it may be desirable to present a user with a projection image having a viewing plane facing directly at the distal tip and another projection image having a viewing plane that optimally displays a curve in the shape of the instrument.

As another example, when generating a projection image, the control system may store a third-dimension coordinate value associated with the highest-value voxel along each projection line orthogonal to the viewing plane. When a point is selected within a single 2D projection image, the control system may retrieve the third-dimension coordinate value associated with the selected point from memory. For example, a projection image having pixels arranged along an X-Y viewing plane may be displayed to a user for selection of a point on the instrument. The pixel corresponding to the selected point on the instrument may be identified, and the X-coordinate and Y-coordinate are determined by the location of the pixel within the viewing plane. A Z-coordinate associated with the voxel which was selected and mapped to that pixel when generating the projection image may be retrieved from memory.

At process 220, the instrument or a portion thereof may be segmented from the intra-operative image data. In this regard, the point identified in process 218 may be used as a seed point and adjacent voxels of the image data having the same or similar intensity values as the selected point may be aggregated to form a 3D shape corresponding to that of the instrument. For example, during the segmentation process, the voxels may be partitioned into segments or elements or may be tagged to indicate that they share certain characteristics or computed properties such as color, density, intensity, and texture. The image data corresponding to the instrument may be segmented from the image data, and a model of the instrument shape may be generated from the voxels partitioned or tagged as being similar to the selected point used to seed the segmentation. For example, the instrument may be identified in the image data by segmentation using an intensity value (e.g., CT number or Hounsfield value) associated with the instrument. This data associated with the instrument may be isolated from other portions of the image data that are associated with the patient or with specific tissue types. A three-dimensional mesh model may be formed around the isolated data and/or a centerline may be determined that represents a centerline of the instrument. The segmented image data for the instrument may be expressed in the intra-operative image reference frame. Morphological operations may be utilized to interconnect non-contiguous aggregated voxels having similar intensity values.

In some examples, segmenting the instrument from the intra-operative image data may include selecting voxels based upon one or more factors including proximity to the selected point, shape data from a shape sensor, an approximate registration of the instrument to the patient, and/or an expected instrument voxel intensity value. An expected instrument voxel intensity value may include a range of values associated with materials from which the instrument is composed. In some examples, an algorithm (e.g., Gaussian Mixture Model) may be used to establish the expected instrument intensity. In some examples, segmenting the instrument from the image data may further comprise utilizing processes established by the control system using deep learning techniques to improve material identification in intra-operative image data.

Known information about properties of the instrument may be used to further seed the segmentation process. For example, an instrument (e.g., a steerable catheter) may include a metal spine embedded in a non-metal sheath. In this regard, high intensity voxels in the intra-operative image data associated with the spine may be identified first, and a region around the spine may be searched for the non-metal sheath in voxels having a lower intensity that the spine. In a similar regard, a high-intensity fiducial marker may be inserted through a working channel of an instrument during intra-operative imaging to improve segmentation of the instrument.

With the instrument identified in the intra-operative image data, it may be desirable to register the intra-operative image data to the instrument to facilitate further functions of the present disclosure. In order to register the intra-operative image data to the instrument, while the intra-operative imaging is performed, shape data from the instrument (e.g., from a shape sensor disposed along a length of the instrument) may be received at a process 222. The shape data may be captured for only a brief period of time during the intra-operative imaging scan or may be captured throughout the image capture period of the intra-operative imaging scan. In order to ensure accurate correlation between shape data and related intra-operative image data, a clock of the instrument control system may be synchronized with a clock of the intra-operative imaging system. In this regard, each timestamped instance of intra-operative image data may be paired with a correspondingly timestamped instance of shape data so that registration may be performed using shape data and intra-operative image data collected at substantially the same time.

At a process 224, the intra-operative image data in the intra-operative image reference frame may be registered to the shape data in the instrument reference frame and/or surgical reference frame by comparing the shape data to the segmented portion of the image data corresponding to the instrument. This registration may rotate, translate, or otherwise manipulate by rigid or non-rigid transforms points associated with the segmented shape and points associated with the shape data. This registration between the model and instrument reference frames may be achieved, for example, by using ICP or another point cloud registration technique. In some examples, the segmented shape of the instrument is registered to the shape data and the associated transform (a vector applied to each of the points in the segmented shape to align with the shape data in the shape sensor reference frame) may then be applied to the entirety of the intra-operative image data (e.g., the anatomy around the segmented instrument) and/or to intra-operative image data subsequently obtained during the medical procedure. The transform may be a six degrees-of-freedom (6DOF) transform, such that the shape data may be translated or rotated in any or all of X, Y, and Z and pitch, roll, and yaw. Optionally, data points may be weighted based upon segmentation confidence or quality to assign more influence to data points which are determined more likely to be accurate. Alternatively, registering the intra-operative image data to the shape data may be performed using coherent point drift or an uncertainty metric (e.g., root-mean-square error).

Discussion of processes for registering an instrument to image data as well as other techniques discussed herein may be found, for example, in International Application Publication No. WO 2021/092116 (filed Nov. 5, 2020) (disclosing “Systems and Methods for Registering an Instrument to an Image Using Change in Instrument Position Data”), International Application Publication No. WO 2021/092124 (filed Nov. 5, 2020) (disclosing Systems and Methods for Registering an Instrument to an Image Using Point Cloud Data), and U.S. Provisional Application No. 63/132,258 (filed Dec. 30, 2020) (disclosing “Systems And Methods For Integrating Intraoperative Image Data With Minimally Invasive Medical Techniques”), all of which are incorporated by reference herein in their entireties.

At a process 226, and as discussed below with reference to FIG. 3B, the segmented instrument shape may be displayed (e.g., overlaid) with the shape data from the instrument (e.g., from a shape sensor within the instrument). By showing the instrument shape from the two sources overlaid one on top of the other, a user may be able to quickly and effectively assess the registration performed in process 224 to confirm its accuracy. While slight or limited deviations of one shape from the other may be deemed acceptable, substantial deviations may indicate an unacceptable registration. In some examples, the instrument shape segmented from the intra-operative image data and the shape data from the instrument may be shown in isolation to a user on a display without patient anatomy or other information which may obscure visual review of the two shapes. In other examples, the two shapes may be shown in the context of one or more projection images generated in process 216 and/or on slices of the intra-operative image data. One or both of the instrument shapes as determined by the shape data and by the segmented shape may be shown with different display properties to distinguish the two instrument shapes.

The control system may be configured to display all or portions of one or both instrument shapes using differing display properties (e.g., color, brightness, opacity, linetype, hatch pattern, thickness, etc.) to provide visual contrast. For example, the segmented shape may be shown in full-thickness corresponding to the diameter of the instrument while the shape data may be shown as a narrow centerline overlaid on the segmented shape. Different regions of the segmented shape may be shown with different properties based on, for example, segmentation confidence levels determined by the control system or image properties (e.g., voxel intensities). Similarly, misaligned portions of one or both instrument shapes may be shown with different display properties to quickly draw a user's attention to potential areas of concern.

A user may be able to manually translate and/or rotate one or both of the instrument shapes to correct a misalignment. For example, a user input may be configured to receive input from a user (e.g., via buttons, knobs, a touchscreen, etc.) and manipulate a selected one of the segmented shape or shape data to manually correct an alignment error arising from the registration. Whether or not a manual correction has been provided, the user may input a confirmation command to the control system upon visually determining the registration is acceptable.

At a process 228, the anatomy adjacent to the instrument may be displayed to aid in identification of the target. That is, at the time the intra-operative image data is collected, the instrument may already be positioned at a deployment location within the anatomy near the location of the target as determined by the 3D model and it may be assumed, therefore, that the distal tip of the instrument is positioned near the target at the time of intra-operative imaging. To facilitate quick identification of the target in the intra-operative image data, the image volume may be truncated by spatially limiting a search space to a truncated region of interest around the distal tip where the target is most likely to be located. It should be appreciated that a tool access port or any other feature of interest along the length of the instrument may be used to establish the truncated region of interest estimated to be near the target based on the location of the instrument with regard to the 3D model instead of the distal tip.

One or more limited projection images may be generated using the region of interest. By limiting the voxels considered in generating the revised projection images to those in a volumetric proximity to the distal tip (or other reference location of the instrument), anatomical structures and tools remote from the distal tip of the instrument may be filtered out and omitted from the limited projection images. In some examples, when the target is not visually discernible in a limited projection image expected to include the target (based on proximity of the instrument to the target in the 3D model), an intensity threshold may be selected and applied to the intra-operative image data within the region of interest. In this regard, a maximum intensity threshold may be selected which filters out high intensity voxels which may be preventing the voxels corresponding to the target from being selected and projected into the viewing plane of the projection image. A maximum intensity threshold used in this manner may be static and pre-programmed in the control system based on anticipated tissue types or may be dynamic. For example, a user may manually adjust the maximum intensity threshold (e.g., using a slider on a user interface), with a revised projection image being displayed with each adjusted threshold, until the target comes into view in the one or more limited projection images. In some examples, a maximum intensity threshold may be automatically selected and/or adjusted based on a variety of factors including, but not limited to, a known property of the target (e.g., tissue density) or a confidence interval of selected and neighboring voxels. It should be appreciated that generating a limited projection image from only a region of interest and/or filtering a projection image by applying a maximum intensity threshold may yield a projection image of the instrument and/or the target having an improved clarity for visual identification of the instrument and/or target. FIG. 3C discussed further below illustrates an example of a limited projection image that has been volumetrically truncated and revised with an intensity threshold.

Additionally, process 228 for displaying the anatomy adjacent to the instrument may include overlaying the instrument shape (e.g., one or more of the segmented shape, a projection image which includes the instrument, or shape data from the instrument) on one or more alternative views instead of or in addition to projection images. For example, an alternative view may include a slice of the intra-operative image data or a projection image having a different applied threshold or truncated volumetric region for providing an additional illustration of the region of interest to aid a user in identifying the target in the intra-operative image data. Overlaying the instrument shape on such an alternative view may assist a user in identifying the target by providing a visual indication of the instrument location with respect to the anatomy. The instrument location may provide a reference on which to base a search space for locating the target. In this regard, the search space to locate the target may be reduced based upon an assumption that the instrument was previously navigated into close proximity with the target so the target should be near the instrument in the alternative view.

At a process 230, the target may be identified in the intra-operative image data. In some examples, identifying the target may include receiving an indication or selection from a user at a user input device. For example, a user may manually select portions of a projection image or alternative view on the display system that are associated with the target. In some examples, the control system may extract the size and shape of the target from the model and overlay a corresponding representation of the target onto the intra-operative image data (e.g., on a projection image or on a slice). For example, an outline or boundary of a 2D profile shape of the target from the perspective of the viewing angle of a particular projection image or slice image may be overlaid on that particular projection image or slice image at its location in the 3D model based on the registration of the 3D model to the instrument and the registration of the intra-operative image data to the instrument as discussed above in relation to processes 210 and 224. In some examples, a target may be represented in the 3D model by an ellipsoid shape such that the outline overlaid on the intra-operative image data will generally be shaped as an ellipse, although more complex 3D shapes and corresponding 2D outlines are contemplated. The representation of the target location from the 3D model overlaid on the intra-operative image data may aid a user in visually identifying the target in the intra-operative image data by providing an anticipated location of the target. Upon identifying the target in the intra-operative image data, the user may manually adjust the size, shape, and/or location of the boundary to more closely correspond to the size and shape of the target in the intra-operative image data. The region within the adjusted boundary may be used by the control system to define the intra-operative size, shape, and location of the target. This procedure may be performed once on a single image or may be repeated over a plurality of images of the intra-operative image data to refine a volumetric size and shape of the target. In some examples, the user may draw a boundary around the target on a number of image planes and the shapes may then be integrated into a mesh or other model structure. In some examples, the target may be automatically segmented from the images.

At a process 232, the intra-operative location (optionally including the size and shape) of the target may be mapped to the instrument reference frame based upon the registration performed in process 224. Further, because the instrument reference frame is registered to the model based upon the registration performed in process 210, the intra-operative location of the target may be mapped to the model. That is, the intra-operative image reference frame may be registered to the image reference frame of the pre-operative image data (and subsequently constructed 3D model) based on their shared registration to the instrument reference frame using the shape of the instrument. In some instances, for example when the target location has not changed between the pre-operative imaging and intra-operative imaging, the target location in the intra-operative image data (e.g., in a projection image, a slice, or other alternative view) may be used for refining registration of the intra-operative image data to the pre-operative image data (or 3D model) based upon a pre-operative location of the target in the model and an intra-operative location of the target in the intra-operative image data.

With the intra-operative target mapped to the 3D model, the intra-operative size, shape, and/or location of the target may be compared to the pre-operative size, shape, and/or location of the target. If there is a meaningful discrepancy, the target in the 3D model may be updated to reflect the intra-operative target at a process 234. For example, the adjusted size, shape, and/or location of one or more target boundaries discussed above in relation to process 230 may be mapped back to the 3D model and used to update the size, shape, and location of the target in the 3D model. The updated target may be shown with respect to the 3D model and/or the instrument shape on the display system to facilitate the procedure, for example, to revise a navigational route and/or a deployment location of the instrument with respect to the target.

FIGS. 3A-3C illustrate an example of a graphical user interface 300 in accordance with the present disclosure. In FIG. 3A three projection images, which may be generated at process 216 of FIG. 2, are displayed in three orthogonal viewing planes—an X-Z view, a Y-Z view, and an X-Y view. These three images illustrate an example of the features which may be visible in a maximum intensity projection image generated from intra-operative volumetric imaging data. As shown, many of the low-density internal organs have been filtered out due to denser materials found along the projection lines orthogonal to each respective viewing plane. For example, bone structures such as the spine 314 and ribcage 316 have produced highest-intensity voxels which have been projected as pixels in the three projection images. Similarly, along the length of the instrument 304, the voxels associated with the instrument have been selected as the highest-intensity voxels and projected into the viewing plane. The distal tip 306 of the instrument 304 may appear as having the greatest contrast to the background due its construction from a high density metal.

As described above in relation to process 218 of FIG. 2, a user may select the distal tip 306, or any other desired point along the instrument 304, in one or more of the displayed projection images via the graphical user interface 300 using an input device. This selection of a point along the instrument, with a coordinate in each of the three dimensions, may be used to seed the instrument segmentation process 220.

FIG. 3B illustrates an example of a graphical user interface 300 after the instrument segmentation process 220 when the shape data 312 has been overlaid on the segmented shape of the instrument 304 as described with reference to process 226. In the illustrated example, a tool 310 (e.g., a biopsy or treatment needle) is shown extended from the distal tip 306 of the instrument at the time the intra-operative image data was captured. A prompt 318 is displayed requesting that the user visually confirm the registration appears to be accurate as shown in each of the three orthogonal projection images. In the illustrated example, the distal tip 306 may be rendered as a blue circle to provide contrast between the location of the distal tip 306 as determined from the shape data and the greyscale image data behind or around it. Similarly, the shape data 312 may be rendered as a blue line to provide contrast between the shape data 312 and the greyscale image data behind or around it. Although the illustrated example references the color blue in the text adjacent to the prompt 318, the distal tip 306 and shape data 312 may be rendered in any suitable color and may be rendered in different colors. Similarly, although the illustrated example references a circle and a line, any suitable graphical clement may be used to visually indicate the respective locations. As will be appreciated by comparing the wide view images of FIG. 3A with the images of FIG. 3B, the projection images of FIG. 3B have been generated with a truncated region of interest around the distal tip 306 as described in relation to process 228 above. By reducing the volumetric region used to generate the projection images of FIG. 3B and/or applying a threshold, the bone structures have been filtered out allowing the target 308 to be projected into the viewing planes. This filtering of the image data in the projection images of FIG. 3B may allow a user or control system to identify the target as discussed above in relation to process 230.

FIG. 3C provides an illustration of a projection image as may be displayed on a graphical user interface 300 after the instrument and target have been identified per processes 218 and 230. As shown in this projection image, the dense bone structures 314 and 316 of the patient and the instrument 304 are clearly visible, despite their three-dimensional shapes. In contrast to slice images generated using the same intra-operative image data, the entire length of the instrument 304 and the distal tip 306 are visible in the intensity-based projection image, allowing a user to quickly and effectively identify the instrument 304 in the image. Additionally, a minimum intensity threshold and/or a maximum intensity threshold may be applied to the region of interest around the distal tip 306 (or around the target 308 after it has been identified), allowing for the target 308 to be displayed without obfuscation from surrounding tissue. For example, despite the ribcage 316 being denser than the target 308, application of a threshold around the distal tip 306 or target 308 may allow the voxels associated with the target 308 to be projected into the projection image in lieu of the voxels associated with the ribcage 316. It will be appreciated that the example illustrated in FIG. 3C may be generated from the same or a different set of intra-operative image data than FIGS. 3A and 3B.

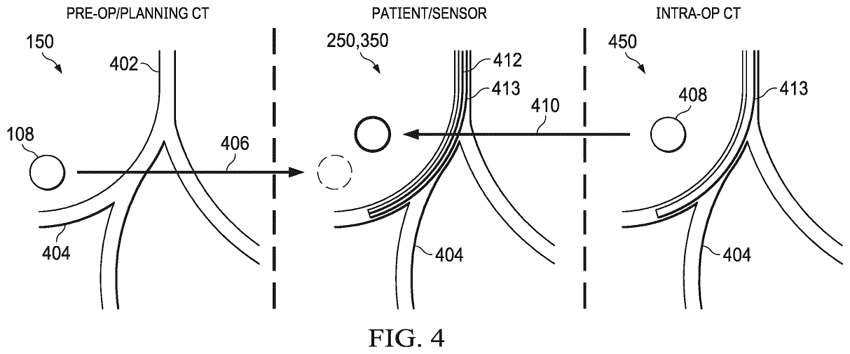

As discussed above in relation to process 210 in FIG. 2, an image reference frame of pre-operative image data may be registered to an instrument reference frame. Similarly, an intra-operative image reference frame may be registered to the instrument reference frame as discussed above in relation to process 224. The common registration between these reference frames allows for updating of the target in the 3D model. FIG. 4 provides a simplified diagram of registering pre-operative image data in an image reference frame 150 and intra-operative image data in an intra-operative image reference frame 450 to shape data 412 from an instrument 413 in an instrument reference frame 350 (which may also be registered to a surgical reference frame 250 in which a patient is positioned). Initially, a 3D model 402 may be constructed from pre-operative image data. The model may include anatomical passageways 404 and a pre-operative location of target 108 disposed relative to anatomical passageways 404. During a medical procedure, an instrument including a shape sensor may be inserted into anatomical passageways 404. Based on the shape of anatomical passageways 404 in the pre-operative image data and shape data 412 from the shape sensor, the image reference frame 150 may be registered to the instrument reference frame 350. Additionally, while the instrument 413 is disposed within anatomical passageways 404, intra-operative imaging may be obtained, for example, using cone beam CT. The intra-operative image data may indicate a target location 408 relative to the instrument in the intra-operative image reference frame 450. Using the shape of the instrument 413 in the intra-operative image data and the shape data 412 from the shape sensor, the intra-operative image reference frame 450 may be registered to the instrument reference frame 350. Accordingly, the image reference frame 150 and the intra-operative image reference frame 450 may also be registered. This registration arrangement allows for the pre-operative location of the target 108 to be updated to the intra-operative target location 408 as described above with reference to FIG. 2. Arrows 406 and 410 illustrate where the pre-operative target location 108 and intra-operative target location 408 indicate the target is located within the surgical reference frame 250. As will be appreciated, the pre-operative target location 108 may provide an outdated or otherwise incorrect location of the target.

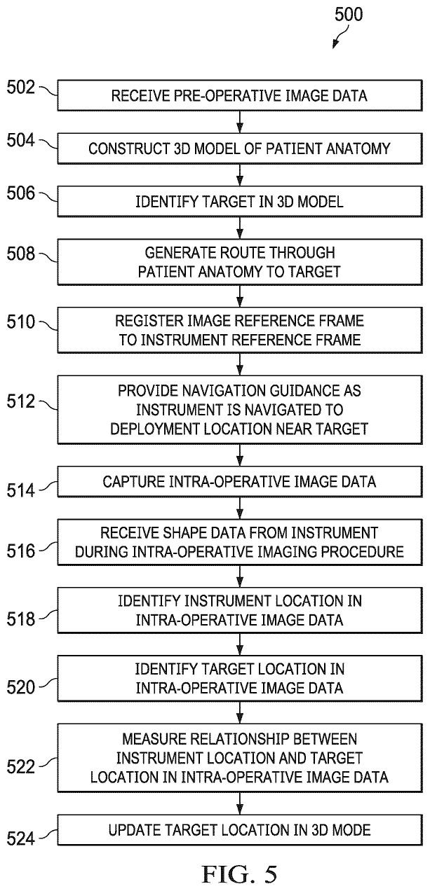

FIG. 5 illustrates an example of a method or workflow 500 for performing a minimally invasive procedure using a non-integrated imaging system in accordance with some aspects of the present disclosure. In this example, intra-operative image data may be used to update a target location using a relationship between an instrument location and a target location measured in the intra-operative image data. It should be appreciated that processes 502-512 are substantially similar to processes 202-212, respectively, of workflow 200 and are only omitted in this description of FIG. 5 to avoid unnecessarily repeating their description. At a process 514, intra-operative image data may be captured by an intra-operative imaging system. The intra-operative image data may include patient anatomy and the instrument disposed within the patient anatomy. At process 516, shape data from the instrument may be received at a control system associated with the instrument during, or in close temporal proximity to, capturing of the intra-operative image data. For example, a shape sensor (e.g., a fiber optic shape sensor or one or more position sensors) disposed along a length of the instrument may be used to provide real-time shape data (e.g., information regarding a shape of the instrument and/or a position of one or more points along the length of the instrument).

At process 518, a location of the instrument may be identified within the intra-operative image data. The location may be identified as a point on the instrument, such as the distal tip or any other suitable location along the length of the instrument. This process may be performed automatically by image processing associated with the intra-operative imaging system or manually by a user selecting a point on the instrument using an input device of the intra-operative imaging system. Similarly, at process 520, a location of the target may be identified within the intra-operative image data. The location may be identified as point within the target, such as a center of mass, a point on an external surface of the target, point on the target closest to the instrument, or any other suitable location within the volume of the target. This process may be performed automatically by image processing associated with the intra-operative imaging system or manually by a user selecting one or more points of the target using an input device of the intra-operative imaging system.

At process 522, a spatial relationship between the identified location of the instrument and the identified location of the target is measured or calculated. This spatial relationship may include a distance, an orientation, or both. The spatial relationship may be measured between a 3D position of the distal tip of the instrument and a 3D position of a point of the target but any suitable location along the instrument may be used which is identifiable in the shape data from the instrument as will be appreciated based on the example discussed below in relation to process 524.

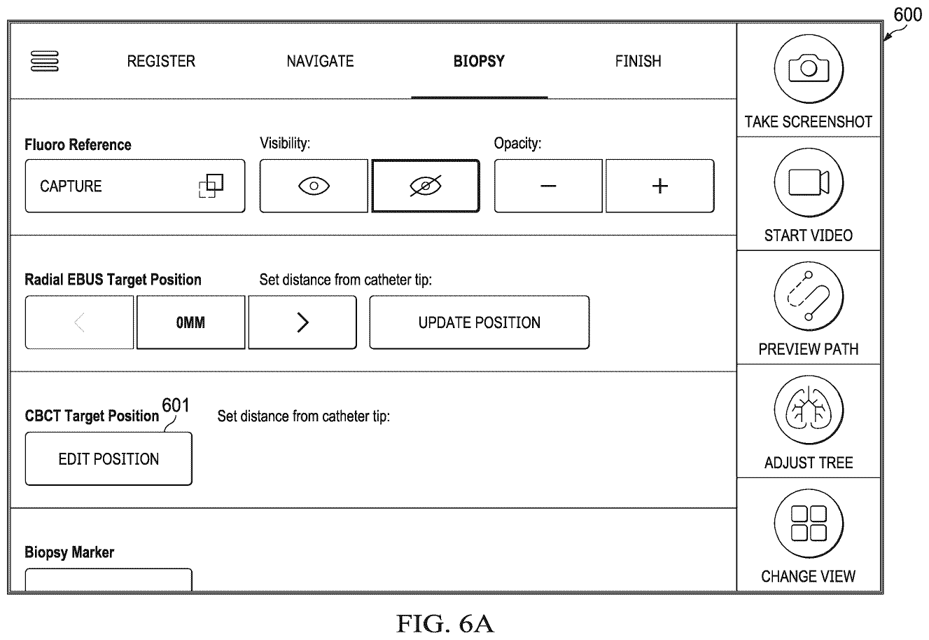

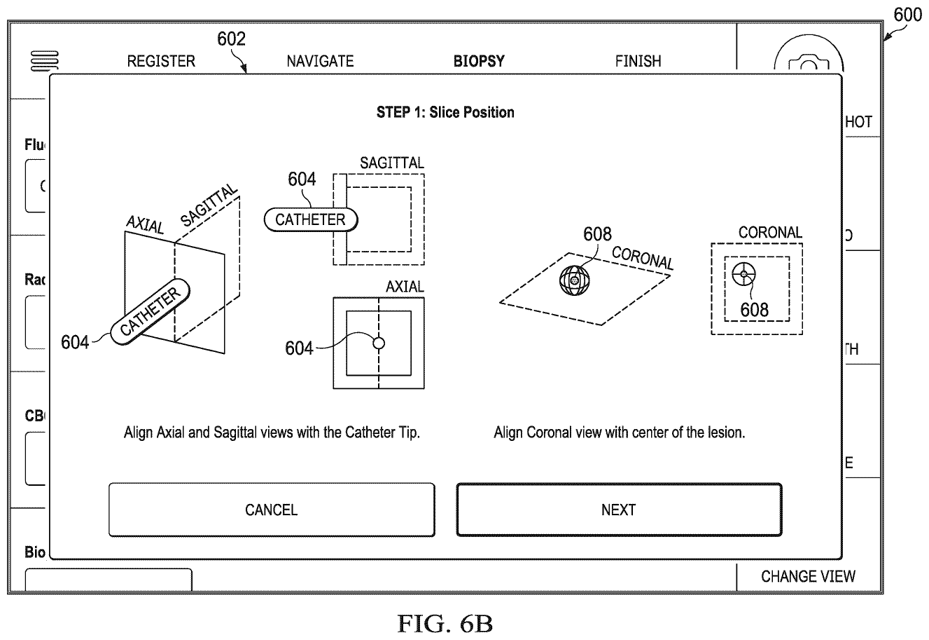

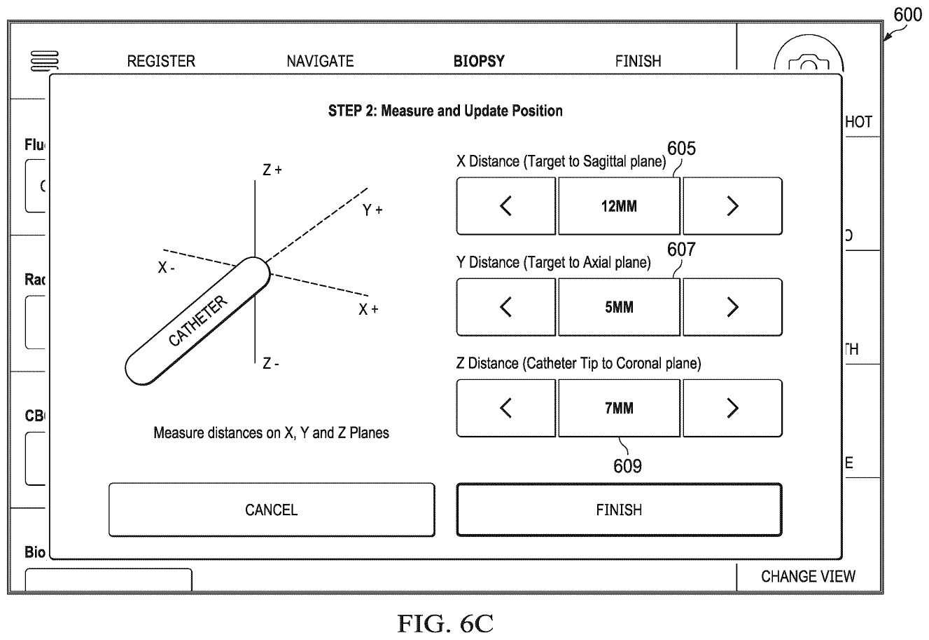

At process 524, a location of the target in the 3D model may be updated based on the spatial relationship between the instrument and target measured at process 522. In some examples, when using a non-integrated imaging system in which the intra-operative imaging system is not in operative communication with the instrument control system for transfer of the imaging data, the spatial relationship may be measured in the intra-operative image data using an interface associated with the intra-operative imaging system. Distance and orientation information defining the spatial relationship may be presented to a user via a display system of the intra-operative imaging system. The instrument control system may be configured to receive one or more user inputs providing an indication of the spatial relationship which may be used to revise a location of the target in the 3D model. In some examples, the spatial relationship may be designated by three offsets (e.g., an X-offset, a Y-offset, and a Z-offset) representing the location of the target with respect to the distal tip, or another location, of the instrument. A user may input these offsets into the instrument control system (as discussed in relation to FIG. 6C below), which in turn, may revise a location of the target in the 3D model to reflect a corresponding position based on the location of the distal tip of the instrument in as indicated by the shape data from the instrument (which is registered to the model as discussed in relation to process 210).

It should be appreciated that the spatial relationship between the instrument and the target may be designated by any suitable means, including one or more of an orientation, an azimuth, an altitude, and/or a distance of any portion of the target with respect to a pose or position of any portion of the instrument (e.g., the distal tip or a tool access port). Generally, a distal end of a shape sensor will coincide with a distal tip of the instrument or have a known and fixed relation thereto such that the location of the distal tip is determinable based on the distal end of the shape data provided by the shape sensor. Similarly, because a shape sensor may be fixed along the length of the instrument, the location of any point along the instrument may be determinable using the shape data. For example, a tool access port opening from a side surface of the instrument may have a known and fixed relation to a point along the shape sensor such that the position of the access port can be determined based on the shape data. As long as the tool access port is visible and identifiable in the intra-operative image data, the tool access port may be selected for measuring the spatial relationship between the instrument and the target in a similar manner described above in relation to the distal tip. It is further contemplated that any other structural feature of an instrument that is identifiable in intra-operative image data (including but not limited to an endoscopic camera, an imaging transducer, a suction or irrigation port, an electromagnetic sensor, a wrist joint, a radio-frequency ablation generator, etc.) may be used in a similar manner.

FIGS. 6A-6C illustrate a graphical user interface 600 incorporating features of the present disclosure, particularly with reference to process 500. FIG. 6A illustrates a menu that may be displayed on a display system (which may optionally include a touchscreen interface) of a control system which stores or communicatively accesses a 3D model of the patient anatomy and the shape data from the instrument. A user may select the button 601 to initiate an update of the target location in the 3D model.

An instruction prompt 602 may be displayed by the display system to guide a user in measuring the spatial relationship between the instrument and the target in the intra-operative image data (as described in relation to process 522 above). In the example instruction prompt 602 shown, the user is provided guidance to align one or more imaging planes (e.g., an axial plane and a sagittal plane) with the distal tip of the catheter 604 and one or more imaging planes (e.g., a coronal plane) with the center of the target 608. As an example, this process may be performed by scrolling through slice images of the intra-operative image data on a user interface of the intra-operative imaging system until the distal tip of the catheter is visible in the sagittal and axial slice views and the center of the target is visible on the coronal slice view. The user interface 600 may then be used to measure, calculate, or otherwise determine the X-distance between target and sagittal plane, the Y-distance between the target and the axial plane, and the Z-distance between the distal tip of the instrument and the coronal plane. Using the X-distance field 605, Y-distance field 607, and Z-distance field 609 displayed on the graphical user interface 600 shown in FIG. 6C, these distances may be input into the control system and the location of the target in the 3D model may updated to reflect a corresponding set of X-, Y-, and Z-distances from the current location of the distal tip of the instrument within the 3D model as indicated by the shape data from the instrument.

It should be appreciated that although FIGS. 6B and 6C describe an example for updating the target location based on the distal tip of the instrument and the center of the target, any suitable point of the instrument and of the target may be used in a similar manner as discussed above. Similarly, although FIG. 6C describes an example using X-, Y-, and Z-distances, the spatial relationship between the instrument and the target may be designated and input into the graphical user interface by any suitable means.

In some examples, the techniques of this disclosure, such as those discussed above in relation to FIGS. 2 and 5, may be used in an image-guided medical procedure performed with a robot-assisted medical system as shown in FIGS. 7A-8. FIG. 7A illustrates a clinical system 10 includes a robot-assisted medical system 700 and an intra-operative imaging system 718. The robot-assisted medical system 700 generally includes a manipulator assembly 702 for operating a medical instrument system 704 (including, for example, instrument 104) in performing various procedures on a patient P positioned on a table T in a surgical environment 701. The manipulator assembly 702 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. A master assembly 706, which may be inside or outside of the surgical environment 701, generally includes one or more control devices for controlling manipulator assembly 702. Manipulator assembly 702 supports medical instrument system 704 and may optionally include a plurality of actuators or motors that drive inputs on medical instrument system 704 in response to commands from a control system 712. The actuators may optionally include drive systems that when coupled to medical instrument system 704 may advance medical instrument system 704 into a naturally or surgically created anatomic orifice. Other drive systems may move the distal portion of medical instrument system 704 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical instrument system 704 for grasping tissue in the jaws of a biopsy device and/or the like.

Robot-assisted medical system 700 also includes a display system 710 (which may the same as display system 100) for displaying an image or representation of the surgical site and medical instrument system 704 generated by a sensor system 708 and/or an endoscopic imaging system 709. Display system 710 and master assembly 706 may be oriented so operator O can control medical instrument system 704 and master assembly 706 with the perception of telepresence.

In some examples, medical instrument system 704 may include components for use in surgery, biopsy, ablation, illumination, irrigation, or suction. Optionally medical instrument system 704, together with sensor system 708 may be used to gather (e.g., measure) a set of data points corresponding to locations within anatomic passageways of a patient, such as patient P. In some examples, medical instrument system 704 may include components of the imaging system 709, which may include an imaging scope assembly or imaging instrument that records a concurrent or real-time image of a surgical site and provides the image to the operator or operator O through the display system 710. The concurrent image may be, for example, a two or three-dimensional image captured by an imaging instrument positioned within the surgical site. In some examples, the imaging system components that may be integrally or removably coupled to medical instrument system 704. However, in some examples, a separate endoscope, attached to a separate manipulator assembly may be used with medical instrument system 704 to image the surgical site. The imaging system 709 may be implemented as hardware, firmware, software or a combination thereof which interact with or are otherwise executed by one or more computer processors, which may include the processors of the control system 712.

The sensor system 708 may include a position/location sensor system (e.g., an electromagnetic (EM) sensor system) and/or a shape sensor system for determining the position, orientation, speed, velocity, pose, and/or shape of the medical instrument system 704.

Robot-assisted medical system 700 may also include control system 712. Control system 712 includes at least one memory 716 and at least one computer processor 714 for effecting control between medical instrument system 704, master assembly 706, sensor system 708, endoscopic imaging system 709, and display system 710. Control system 712 also includes programmed instructions (e.g., a non-transitory machine-readable medium storing the instructions) to implement some or all of the methods described in accordance with aspects disclosed herein, including instructions for providing information to display system 710.

Control system 712 may optionally further include a virtual visualization system to provide navigation assistance to operator O when controlling medical instrument system 704 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

An intra-operative imaging system 718 may be arranged in the surgical environment 701 near the patient P to obtain images of the patient P during a medical procedure. The intra-operative imaging system 718 may provide real-time or near real-time images of the patient P. In some examples, the intra-operative imaging system 718 may be a mobile C-arm cone-beam CT imaging system for generating three-dimensional images. For example, the intra-operative imaging system 718 may be a DynaCT imaging system from Siemens Corporation of Washington, D.C., or other suitable imaging system. In other examples, the imaging system may use other imaging technologies including CT, MRI, fluoroscopy, thermography, ultrasound, optical coherence tomography (OCT), thermal imaging, impedance imaging, laser imaging, nanotube X-ray imaging, and/or the like. The example clinical system 10 of FIG. 7A illustrates the intra-operative imaging system 718 being in operative communication with the control system 712 for transferring the intra-operative image data to the control system, as would be the case in an integrated imaging system, as that term is used herein. It should be appreciated that the present disclosure also contemplates a non-integrated imaging system in which the intra-operative imaging system 718 is not configured to transfer the intra-operative image data to the control system 712 and may include a display system for displaying the intra-operative image data to a user.

FIG. 7B provides a simplified illustration of communication between the control system 712 and the intra-operative imaging system 718 as may be used in an integrated imaging system. In some examples, the control system 712 includes a processor 714, a memory 716, a communication device 720, and a clock 722. Although the control system 712 is shown as a single block in the simplified schematics of FIGS. 7A and 7B, the control system 712 may include multiple processors, memories, communication devices, and clocks. Furthermore, the components of the control system 712 may be distributed throughout the medical system 700, including at the manipulator assembly 702, the instrument system 704 and the master assembly 706. In some examples, the intra-operative imaging system includes a processor 724, a memory 726, a communication device 728, and a clock 730. The processor 724 is configured to execute programmed instructions stored, for example, on memory 726 to implement some or all of the methods described in accordance with aspects disclosed herein. The clocks 722, 730 may include any type of digital clock, analog clock, software-based clock, or other timekeeping device. The communication devices 720, 728 may include information transmitters, information receivers, information transceivers or a combination of transmitting or receiving devices that enable wired or wireless communication between the imaging system 718 and the control system 712 and/or between the clocks 722, 730. The communication devices 720, 728 may be used to exchange information between the two systems including, for example, clock signals, start and stop signals, image data, patient data, and sensor data.

FIG. 8 illustrates a surgical environment 800 with a surgical reference frame (X_{S}, Y_{S}, Z_{S}) 250 in which the patient P is positioned on the table T. Patient P may be stationary within the surgical environment in the sense that gross patient movement is limited by sedation, restraint, and/or other means. Cyclic anatomic motion including respiration and cardiac motion of patient P may continue unless the patient is asked to hold his or her breath to temporarily suspend respiratory motion. Within surgical environment 800, a medical instrument 804 (e.g., the medical instrument system 704), having a medical instrument reference frame (X_{M}, Y_{M}, Z_{M}) 350, is coupled to an instrument carriage 806. In this example, medical instrument 804 includes an elongate device 810, such as a flexible catheter, coupled to an instrument body 812. Instrument carriage 806 is mounted to an insertion stage 808 fixed within surgical environment 800. Alternatively, insertion stage 808 may be movable but have a known location (e.g., via a tracking sensor or other tracking device) within surgical environment 800. In these alternatives, the medical instrument reference frame is fixed or otherwise known relative to the surgical reference frame. Instrument carriage 806 may be a component of a robot-assisted manipulator assembly (e.g., robot-assisted manipulator assembly 802) that couples to medical instrument 804 to control insertion motion (i.e., motion along an axis A) and, optionally, motion of a distal portion 818 of the elongate device 810 in multiple directions including yaw, pitch, and roll. Instrument carriage 806 or insertion stage 808 may include actuators, such as servomotors, (not shown) that control motion of instrument carriage 806 along insertion stage 808.

In this example, a sensor system (e.g., sensor system 708) includes a shape sensor 814. Shape sensor 814 may include an optical fiber extending within and aligned with elongate device 810. In one example, the optical fiber has a diameter of approximately 200 μm. In other examples, the dimensions may be larger or smaller. The optical fiber of shape sensor 814 forms a fiber optic bend sensor for determining the shape of the elongate device 810. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fiber Bend Sensor”), which are all incorporated by reference herein in their entireties. Sensors in some examples may employ other suitable strain sensing techniques, such as Rayleigh scattering, Raman scattering, Brillouin scattering, and Fluorescence scattering. In some examples, the shape of the catheter may be determined using other techniques. For example, a history of the distal portion pose of elongate device 810 can be used to reconstruct the shape of elongate device 810 over the interval of time.

As shown in FIG. 8, instrument body 812 is coupled and fixed relative to instrument carriage 806. In some examples, the optical fiber shape sensor 814 is fixed at a proximal point 816 on instrument body 812. In some examples, proximal point 816 of optical fiber shape sensor 814 may be movable along with instrument body 812 but the location of proximal point 816 may be known (e.g., via a tracking sensor or other tracking device). Shape sensor 814 measures a shape from proximal point 816 to another point such as distal portion 818 of elongate device 810 in the medical instrument reference frame (X_{M}, Y_{M}, Z_{M}) 350.

Elongate device 810 includes a channel (not shown) sized and shaped to receive a medical tool 822. In some examples, medical tool 822 may be used for procedures such as surgery, biopsy, ablation, illumination, irrigation, or suction. Medical tool 822 can be deployed through elongate device 810 and used at a target location within the anatomy. Medical tool 822 may include, for example, image capture probes, biopsy instruments, laser ablation fibers, and/or other surgical, diagnostic, or therapeutic tools. Medical tool 822 may be advanced from the distal portion 818 of the elongate device 810 to perform the procedure and then retracted back into the channel when the procedure is complete. Medical tool 822 may be removed from a proximal end of elongate device 810 or from another optional instrument port (not shown) along elongate device 810.

Elongate device 810 may also house cables, linkages, or other steering controls (not shown) to controllably bend distal portion 818. In some examples, at least four cables are used to provide independent “up-down” steering to control a pitch of distal portion 818 and “left-right” steering to control a yaw of distal portion 818.

A position measuring device 820 provides information about the position of instrument body 812 as it moves on insertion stage 808 along an insertion axis A. Position measuring device 820 may include resolvers, encoders, potentiometers, and/or other sensors that determine the rotation and/or orientation of the actuators controlling the motion of instrument carriage 806 and consequently the motion of instrument body 812. In some examples, insertion stage 808 is linear, while in other examples, the insertion stage 808 may be curved or have a combination of curved and linear sections.

An intra-operative imaging system 830 (e.g., imaging system 718) is arranged near the patient P to obtain three-dimensional images of the patient while the elongate device 810 is extended within the patient. The intra-operative imaging system 830 may provide real-time or near real-time images of the patient P.

In some examples, the medical instrument 804 or another component of a robot-assisted medical system registered to the medical instrument 804 may include an instrument clock 824. The imaging system 830 may include an imaging clock 826. The clocks 824, 826 may be time synchronized on a predetermined schedule or in response to a synchronization initiation event generated by a user, a control system, or a synchronization system. In some examples, the clocks 824, 826 may be components of a synchronization system that may be a centralized or distributed system further comprising servers, wired or wireless communication networks, communication devices, or other components for executing synchronization algorithms and protocols. In some examples, the medical instrument 804 or another component of a robot-assisted medical system registered to the medical instrument 804 may include a communication device 828. The imaging system 830 may include a communication device 832. The medical instrument 804 and the imaging system 830 may exchange data via their respective communications devices.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the foregoing description, one or more elements shown and described in association with one example, implementation, or application may be incorporated into other examples, implementations, or application unless specifically described otherwise, unless the one or more elements would make an example or implementation non-functional, or unless two or more of the elements provide conflicting functions. Similarly, it should be understood that any particular element, including a system component or a method process, is optional and is not considered to be an essential feature of the present disclosure unless expressly stated otherwise.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

While some examples are provided herein in the context of medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques may also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 712) or may be implemented, at least in part, in the form of executable code stored on non-transitory, tangible, machine-readable media that when run by one or more processors (e.g., the processors 714 of control system 712) may cause the one or more processors to perform one or more of the processes. The terms “a processor” or “the processor” as used herein may encompass a processing unit that includes a single processor or two or more processors.

One or more elements in examples of this disclosure may be implemented in software to execute on a processor of a computer system such as control processing system. When implemented in software, the elements of the examples of the present disclosure are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In one example, the control system supports wireless communication protocols such as Bluetooth, IrDA, HomeRF, IEEE 802.11, DECT, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the present disclosure are not described with reference to any particular programming language. It will be appreciated that a variety of programming languages may be used to implement the teachings of the present disclosure.

In some instances, well-known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along a length of an object.

While certain illustrative examples of the present disclosure have been described and shown in the accompanying drawings, it is to be understood that such examples are merely illustrative of and not restrictive on the broad disclosure herein, and that the examples of the present disclosure should not be limited to the specific constructions and arrangements shown and described, since various other modifications may occur to those ordinarily skilled in the art.

1. A system comprising:
a processor;
a display; and
a memory having computer readable instructions stored thereon that, when executed by the processor, cause the system to:
receive intra-operative three-dimensional image data from an imaging system, wherein a portion of the intra-operative three-dimensional image data corresponds to an instrument disposed in a patient anatomy;
generate two-dimensional projection image data from the intra-operative three-dimensional image data;
display the two-dimensional projection image data on the display; and
identify, within the two-dimensional projection image data, a three-dimensional location of a portion of the instrument.

2. The system of claim 1, wherein the computer readable instructions, when executed by the processor, cause the system to:
segment, based on the identified three-dimensional location of the portion of the instrument, the portion of the intra-operative three-dimensional image data corresponding to the instrument.

3. The system of claim 2, wherein the computer readable instructions, when executed by the processor, cause the system to:
register the intra-operative three-dimensional image data to shape data from the instrument by comparing the shape data to the portion of the intra-operative three-dimensional image data corresponding to the instrument; and
display a two-dimensional projection of the shape data on the two-dimensional projection image data.

4. The system of claim 3, wherein the computer readable instructions, when executed by the processor, cause the system to:
identify one or more regions of the shape data that is misaligned with the portion of the intra-operative three-dimensional image data corresponding to the instrument; and
display the one or more regions with at least one visual property different than one or more regions of the shape data that is aligned with the portion of the intra-operative three-dimensional image data corresponding to the instrument.

5. The system of claim 4, wherein the at least one visual property comprises at least one of a color, a brightness, a linetype, a pattern, or an opacity.

6. The system of claim 3, wherein the computer readable instructions, when executed by the processor, cause the system to:
receive a user input; and
based on the user input, adjust at least one of a position or a rotation of the shape data with respect to the intra-operative three-dimensional image data.

7. The system of claim 1, wherein the computer readable instructions, when executed by the processor, cause the system to:
generate a model of the patient anatomy based on pre-operative image data; and
update the model based on the intra-operative three-dimensional image data.

8. The system of claim 7, wherein updating the model comprises revising a location of an anatomical target.

9. The system of claim 8, wherein the computer readable instructions, when executed by the processor, cause the system to:
generate a navigation path through the patient anatomy based on the pre-operative image data, and wherein updating the model comprises revising the navigation path to correspond to the revised location of the anatomical target.

10. The system of claim 1, wherein the computer readable instructions, when executed by the processor, cause the system to:
generate a model of the patient anatomy based on pre-operative image data; and
register the model to the intra-operative three-dimensional image data based at least in part on a location of an anatomical target in each of the model and the intra-operative three-dimensional image data.

11. The system of claim 1, wherein the computer readable instructions, when executed by the processor, cause the system to:
extract a three-dimensional boundary of an anatomical target from a model of the patient anatomy generated based on pre-operative image data; and
display a projection of the three-dimensional boundary of the anatomical target on the two-dimensional projection image data.

12. The system of claim 11, wherein the computer readable instructions, when executed by the processor, cause the system to:
receive an input from a user to manipulate at least one of a location or a dimension of the projection of the three-dimensional boundary.

13. The system of claim 1, wherein the imaging system comprises a cone-beam computed tomography system.

14. The system of claim 1, wherein the two-dimensional projection image data comprises at least one maximum intensity projection of the intra-operative three-dimensional image data based on voxel intensity values.

15. The system of claim 14, wherein displaying the two-dimensional projection image data on the display comprises displaying a plurality of views with different view orientations.

16. The system of claim 15, wherein the plurality of views comprises at least a first view and a second view, wherein an orientation of the first view is orthogonal to an orientation of the second view.

17. (canceled)

18. The system of claim 16, wherein identifying the three-dimensional location of the portion of the instrument comprises:
receiving a first user input indicating a first two-dimensional location of the portion of the instrument in the first view; and
receiving a second user input indicating a second two-dimensional location of the portion of the instrument in the second view.

19. The system of claim 1, wherein the computer readable instructions, when executed by the processor, cause the system to:
select a region of interest within the intra-operative three-dimensional image data based on the identified three-dimensional location of the portion of the instrument;
generate two-dimensional projection image data from the selected region of interest within the intra-operative three-dimensional image data; and
display the two-dimensional projection image data from the selected region of interest on the display.

20. The system of claim 1, wherein displaying the two-dimensional projection image data on the display comprises displaying a first view with a view plane having a view plane orientation, and wherein identifying the three-dimensional location of the portion of the instrument comprises:
receiving a user input indicating a location of the portion of the instrument in the first view, wherein the indicated location is identifiable by a first coordinate value and a second coordinate value of respective orthogonal first and second axes within the view plane; and
identifying a third coordinate value associated with the indicated location by retrieving a stored coordinate value of a voxel producing a maximum intensity at the indicated location of the portion of the instrument in the first view, wherein the third coordinate value represents an axis orthogonal to the view plane orientation.

21-24. (canceled)

25. A method comprising:
registering shape data from an instrument disposed in a patient anatomy to a model of the patient anatomy, wherein the model of the patient anatomy includes an anatomical target;
displaying the shape data in relation to the model of the patient anatomy on a display;
obtaining intra-operative three-dimensional image data with an imaging system, wherein the intra-operative three-dimensional image data includes at least a portion of the instrument and the anatomical target;
measuring a relationship between the portion of the instrument and the anatomical target in the intra-operative three-dimensional image data; and
revising a location of the anatomical target in the model of the patient anatomy so that a relationship between a portion of the shape data corresponding to the portion of the instrument and the location of the anatomical target in the model of the patient anatomy corresponds to the measured relationship between the portion of the instrument and the anatomical target in the intra-operative three-dimensional image data.

26-28. (canceled)
